(19) United States
(12) Patent Application Publication
(10) Pub. No.: US 2024/0283892 A1
(43) Pub. Date: Aug. 22, 2024

(54) SYSTEM AND METHOD FOR DISPLAYING A SEQUENCE OF IMAGE FRAMES AS A CINE-LOOP WITH MULTIPLE PLAYBACK SPEEDS

(71) Applicant: GE Precision Healthcare LLC, Wauwatosa, WI (US)

(72) Inventor: Svein Arne Aase, Trondheim (NO)

(21) Appl. No.: 18/170,283

(22) Filed: Feb. 16, 2023

(51) Int. Cl. H04N 5/783; A61B 8/08; G06T 3/40

(52) U.S. Cl. CPC H04N 5/783; A61B 8/0883; G06T 3/40

(57) ABSTRACT

A system and method for displaying a sequence of image frames as a cine-loop with multiple playback speeds. The system and method includes accessing a sequence of image frames representing at least one cardiac cycle. The system and method includes identifying a target sequence of image frames including a cardiac event, wherein the target sequence of image frames is a subset of the sequence of image frames. The system and method includes displaying the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed.

### FIELD OF THE INVENTION

This disclosure relates generally to an ultrasound imaging system and a method for displaying a sequence of image frames as a cine-loop with multiple playback speeds.

### BACKGROUND OF THE INVENTION

Echocardiography is commonly used to acquire a sequence of image frames of ultrasound data from a patient's heart in order to evaluate the cardiac behavior around specific cardiac events, such as the opening or closing of a valve. With conventional ultrasound imaging systems and conventional methods of echocardiography, a clinician will typically acquire a sequences of image frames over a period of time equal to or greater than a single heart cycle. The clinician will then oftentimes use the conventional ultrasound imaging system to display the sequence of image frames of ultrasound data on a display device as a cine-loop. Viewing the sequence of image frames as a cine-loop enables the clinician to evaluate the performance and behavior of the patient's heart around one or more desired cardiac events.

A shortcoming of conventional ultrasound imaging systems and conventional methods of echocardiography is that the clinician is commonly only interested in a fraction of the image frames represented in the cine-loop. For example, the clinician may only be interested in the image frames that represent a particular cardiac event (such as a valve opening or closing) and a brief period of time immediately before the cardiac event and a brief period of time immediately after the cardiac event. However, conventional ultrasound imaging systems and conventional methods of echocardiography do not provide the clinician with and fast and easy way to focus on just the portion of the cine-loop spatially close in time to the cardiac event. Some conventional ultrasound imaging systems provide the clinician with the ability to view just one particular phase, such as the systolic phase or the diastolic phase of the cardiac cycle, but these solution still do not provide the clinician with a fast and easy way to focus on just the portion of the cine-loop that is spatially close in time to the cardiac event. The clinician may want to reduce the playback speed of the cine-loop in order to study the behavior of the cardiac event in great detail. However, using conventional ultrasound imaging systems and conventional methods of echocardiography, the clinician is forced to reduce the playback speed of the entire cine-loop. Reducing the speed of the entire cine-loop makes it harder for the clinician to intuitively understand which portion of the cine-loop is the most relevant for the current evaluation/diagnosis. Furthermore, reducing the playback speed of the entire cine-loop increases the total amount of time that it takes for the cine-loop to play.

For at least the reasons discussed hereinabove, there is a need for an improved ultrasound imaging system and method of echocardiography to allow for the identification of a target sequence of image frames including a desired cardiac event, and to enable the display of the sequence of image frames on a display device as a cine-loop with multiple playback speeds so the target sequence of image frames may be displayed at a slower playback speed.

### BRIEF DESCRIPTION OF THE INVENTION

The above-mentioned shortcomings, disadvantages and problems are addressed herein which will be understood by reading and understanding the following specification.

In an embodiment, a method of echocardiography includes accessing a sequence of image frames representing at least one cardiac cycle, wherein each of the image frames in the sequence of image frames is based on ultrasound data. The method includes identifying a target sequence of image frames including a cardiac event, wherein the target sequence of image frames is a subset of the sequence of image frames. The method includes displaying the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed.

In an embodiment, an ultrasound imaging system includes an ultrasound probe, a display device, and at least one processor in electronic communication with the ultrasound probe and the display device, wherein the at least one processor is configured to access a sequence of image frames representing at least one cardiac cycle, wherein each of the image frames in the sequence of image frames is based on ultrasound data, identify a target sequence of image frames including a cardiac event, wherein the target sequence of image frames is a subset of the sequence of image frames, and display the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart of a method in accordance with an exemplary embodiment;

FIG. 3 is a schematic representation of a sequence of image frames with respect to a timeline in accordance with an exemplary embodiment;

FIG. 4 is a representation of an electrocardiogram (ECG) waveform shown with respect to the timeline in accordance with an embodiment;

FIG. 5 is a representation of a ECG waveform and a timeline in accordance with an exemplary embodiment;

FIG. 6 is a graph of playback speed versus time according to an exemplary embodiment;

FIG. 7 is a representation of an ECG waveform and a timeline according to an exemplary embodiment;

FIG. 8 is a graph of playback speed versus time according to the embodiment;

FIG. 9 is a flow chart of a method in accordance with an exemplary embodiment;

FIG. 10 is a schematic diagram of a neural network in accordance with an exemplary embodiment; and

FIG. 11 is a representation showing input and output connections for a neuron of a neural network in accordance with an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized, and that logical, mechanical, electrical and other changes may be made without departing from the scope of the embodiments. The following detailed description is, therefore, not to be taken as limiting the scope of the invention.

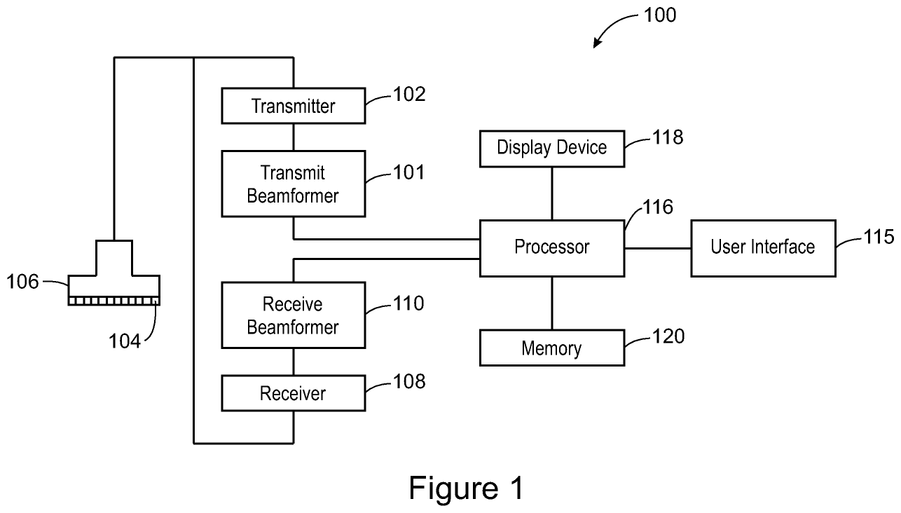

FIG. 1 is a schematic diagram of an ultrasound imaging system 100 in accordance with an embodiment. The ultrasound imaging system 100 includes a transmit beamformer 101 and a transmitter 102 that drive elements 104 within an ultrasound probe 106 to emit pulsed ultrasonic signals into a body (not shown) through one or more transmit events. The ultrasound imaging system 100 is configured to perform echocardiography. The ultrasound probe 106 may be any type of ultrasound probe that may be used to perform echocardiography. The ultrasound probe 106 may, for instance be a transthoracic probe or a transesophageal echocardiography probe (TEE probe). The ultrasound probe 106 may be a linear probe, a convex probe, a phased array probe, or any other type of ultrasound probe configured to be used to perform an echocardiography exam. The ultrasound probe may be a 1D array probe, a 1.25D array probe, a 1.5 D array probe, a 1.75D array probe, or a 2D array probe, such as an e4D matrix probe, according to various embodiments. Still referring to FIG. 1, the pulsed ultrasonic signals are back-scattered from structures in the body, like blood cells or muscular tissue, to produce echoes that return to the elements 104. The echoes are converted into electrical signals by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the ultrasound probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, parameters, and the like. The user interface 115 may include one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The user interface 115 is in electronic communication with the processor 116. The processor 116 may include one or more central processing units (CPUs), one or more microprocessors, one or more microcontrollers, one or more graphics processing units (GPUs), one or more digital signal processors (DSPs), and the like. According to some embodiments, the processor 116 may include one or more GPUs, where some or all of the one or more GPUs include a tensor processing unit (TPU). According to embodiments, the processor 116 may include a field-programmable gate array (FPGA), or any other type of hardware capable of carrying out processing functions. The processor 116 may be an integrated component or it may be distributed across various locations. For example, according to an embodiment, processing functions associated with the processor 116 may be split between two or more processors based on the type of operation. For example, embodiments may include a first processor configured to perform a first set of operations and a second, separate processor to perform a second set of operations. According to embodiments, one of the first processor and the second processor may be configured to implement a neural network. The processor 116 may be configured to execute instructions accessed from a memory. According to an embodiment, the processor 116 is in electronic communication with the ultrasound probe 106, the receiver 108, the receive beamformer 110, the transmit beamformer 101, and the transmitter 102. For purposes of this disclosure, the term “electronic communication” may be defined to include both wired and wireless connections. The processor 116 may control the ultrasound probe 106 to acquire ultrasound data. The processor 116 controls which of the elements 104 are active and the shape of a beam emitted from the ultrasound probe 106. The processor 116 is also in electronic communication with a display device 118, and the processor 116 may process the ultrasound data into image frames for display on the display device 118. According to embodiments, the processor 116 may also include a complex demodulator (not shown) that demodulates the RF data and generates raw data. In another embodiment, the demodulation may be carried out earlier in the processing chain. The processor 116 may be adapted to perform one or more processing operations according to a plurality of selectable ultrasound modalities on the data. The data may be processed in real-time during a scanning session as the echo signals are received. The processor 116 may be configured to scan-convert the ultrasound data acquired with the ultrasound probe 106 so it may be displayed on the display device 118 as one or more image frames. Displaying ultrasound data in real-time may involve displaying image frames based on the ultrasound data without any intentional delay. For example, the processor 116 may display each updated image frame as soon as ultrasound data for each respective image frame has been acquired and processed for display during the process of an ultrasound procedure. Real-time frame rates may vary based on the size of the region or volume from which data is acquired and the specific parameters used during the acquisition. According to other embodiments, the data may be stored temporarily in a buffer (not shown) during a scanning session and processed in less than real-time. According to embodiments that include a software beamformer, the functions associated with the transmit beamformer 101 and/or the receive beamformer 108 may be performed by the processor 116.

According to various embodiments, the components illustrated in FIG. 1 may be part of a distributed ultrasound imaging system. For example, one or more of the processor 116, the user interface 115, the transmitter 102, the transmit beamformer 101, the receive beamformer 110, the receiver 108, a memory 120, and the display device 118 may be located remotely from the ultrasound probe 106. The aforementioned components may be located in different rooms or different facilities according to various embodiments. For example, the probe 106 may be used to acquire ultrasound data from the patient and then transmit the ultrasound data, via either wired or wireless techniques, to the processor 116.

According to an embodiment, the ultrasound imaging system 100 may continuously acquire ultrasound data at a frame rate of, for example, 20 Hz to 80 Hz. Image frames generated from the ultrasound data may be refreshed at similar frame rates. Other embodiments may acquire data and display image frames at different rates. For example, some embodiments may acquire ultrasound data at a volume rate of less than 20 Hz or greater than 80 Hz depending on the size of the ultrasound data within each image frame and the parameters associated with the specific application. The memory 120 is included for storing processed image frames. In an exemplary embodiment, the memory 120 is of sufficient capacity to store image frames of ultrasound data acquired over a period of time at least several seconds in length. The image frames may be stored in a manner to facilitate retrieval thereof according to its order or time of acquisition. The memory 120 may comprise any known data storage medium.

In various embodiments of the present invention, data may be processed by other or different mode-related modules by the processor 116 (e.g., B-mode, color flow Doppler, M-mode, color M-mode, spectral Doppler, Elastography, TVI, strain, strain rate, and the like) to form two-dimensional ultrasound data or three-dimensional ultrasound data. For example, one or more modules may generate B-mode, color flow, color Doppler, M-mode, color M-mode, spectral Doppler, Elastography, TVI, strain, strain rate and combinations thereof, and the like. The image beams and/or images are stored, and timing information indicating a time at which the data was acquired may be recorded. The modules may include, for example, a scan conversion module to perform scan conversion operations to convert the ultrasound data for each image frame from beam space coordinates to display space coordinates. A video processor module may be provided that reads the image frames from a memory, such as the memory 120, and displays the image frames in real-time while a procedure is being carried out on a patient. The video processor module may store the image frames in an image memory, from which the image frames are read and displayed.

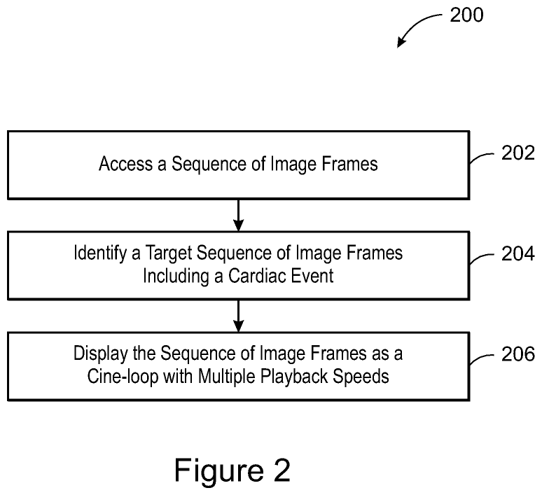

FIG. 2 is a flow chart of a method 200 in accordance with an exemplary embodiment. The individual blocks of the flow chart represent steps that may be performed in accordance with the method 200. Additional embodiments may perform the steps shown in a different sequence and/or additional embodiments may include additional steps not shown in FIG. 2. The technical effect of the method 200 is the displaying of a sequence of image frames as a cine-loop with multiple playback speeds, where a target sequence of image frames is displayed at a slower playback speed. The method 200 may be performed with the ultrasound imaging system 100 shown in FIG. 1. The method 200 will be described in detail hereinafter.

At step 202 of the method 200, the processor 116 accesses a sequence of image frames representing at least one cardiac cycle. Each of the image frames in the sequence of image frames may represent either a two-dimensional (2D) ultrasound image frame or a three-dimensional (3D) ultrasound image frame depending upon the type of ultrasound probe 106 and/or the imaging mode used during the acquisition. According to various other embodiments, some or all of the image frames may include color flow information (i.e., information acquired using a Doppler color flow mode). According to various embodiments, some or all of the image frames may not include color flow information. The processor 116 may access the sequence of image frames directly from the ultrasound probe 106. According to other embodiments, the processor 116 may access the sequence of image frames from a memory or a storage device. For example, the sequence of image frames may have been previously acquired and the processor 116 may access the sequence of image frames from memory or storage located on the ultrasound imaging system, such as the memory 120, or the processor 116 may access the sequence of image frames from a remote memory or a remote storage location. For example, the processor 116 may access the sequence of image frames from a different ultrasound imaging system, from a remote server, from a Picture Archiving and Communications System (PACS), or from any other remote location.

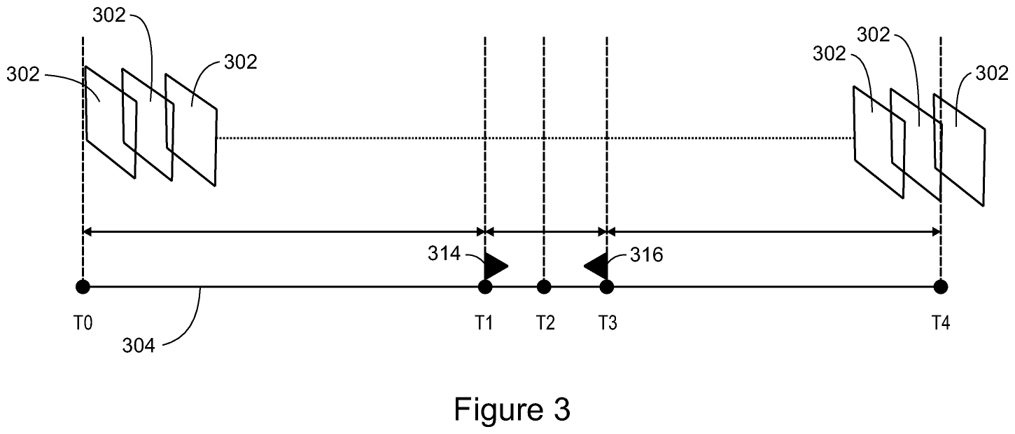

FIG. 3 is a schematic representation of a sequence of image frames with respect to a timeline 304 in accordance with an exemplary embodiment. Each image frame 302 in the sequence of image frames is represented by a perspective view of a rectangle. As discussed previously, each image frame 302 may be generated based on either 2D ultrasound data or 3D ultrasound data. As discussed previously, some or all of the image frames may include color flow information and/or some or all of the image frames may not include color flow information. Color flow information may be included on image frames generated from either 2D ultrasound data or 3D ultrasound data according to various embodiments. According to an embodiment, the sequence of image frames represents at least one cardiac cycle. This means that each image frame 302 was acquired at a different time and, as such, represents a specific cardiac phase. If the sequence of image frames was acquired over a single cardiac cycle, then each image frame 302 represents a unique cardiac phase. However, if the sequence of image frames was acquired over multiple cardiac cycles, each image frame 302 from within a single cardiac cycle represents a unique cardiac phase, but image frames from different cardiac cycles may represent the same cardiac phase.

FIG. 3 schematically represents the sequence of image frames 302 over a single cardiac cycle from a time T0 to a time T4. Each representation of an image frame shown in FIG. 3 is positioned at a different location with respect to the timeline 304 to show the acquisition time associated with that particular image frame. The representation of the image frames shown in FIG. 3 is meant to be schematic and it is to be understood that most sequences of image frames will include significantly more image frames than the number schematically represented in FIG. 3. For example, the ultrasound imaging system may be configured to acquire between 30 and 80 image frames per cardiac cycle. It should, however, be appreciated that the ultrasound imaging system may be configured to acquire more than 80 image frames per cardiac cycle or less than 30 image frames per cardiac cycles according to various embodiments.

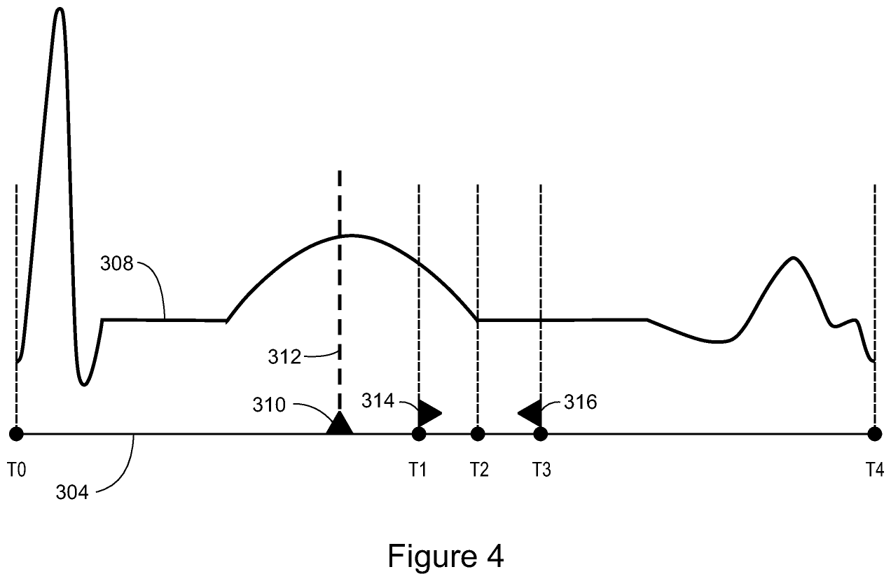

FIG. 4 is a representation of an electrocardiogram (ECG) waveform 308 shown with respect to the timeline 304 in accordance with an embodiment. The timeline 304 shown in FIG. 4 is the same as the timeline 304 shown in FIG. 3. The timeline 304 includes a time T0, a time T1, a time T2, a time T3, and a time T4. The time interval from T0 to the time T4 represents one complete cardiac cycle as evidenced by the ECG waveform 308. The ECG waveform 308 provides an indication of the cardiac phase associated with each of the image frames in the sequence of image frames. According to some embodiments, the ECG waveform 308 and the timeline 304 may be displayed on the display device 118.

Referring back to FIG. 2, at step 204, a target sequence of image frames including a cardiac event is identified. Step 204 may be performed manually, automatically, or semi-automatically. According to various embodiments, a target image frame representing the cardiac event may be identified.

According to an embodiment where the target sequence is identified manually, the operator may manually designate the portion of the sequence of image frames that is the target sequence. For example, the user may designate the portion of the sequence of image frames that is the target sequence using one or more commands input through the user interface 115. For example, the user may use a mouse, a trackpad, a touchpad, a touchscreen, etc. in order to identify the target sequence.

The processor 116 may be configured to display the ECG waveform 308 and the timeline 304 on the display device 118 to represent the sequence of images. According to an exemplary embodiment, the user may use the user interface 115 to control one or both of an image frame displayed on the display device 118 and the manual identification of the target sequence.

For example, the user may use the user interface 115 to position an icon, such as the icon 310 (shown in FIG. 4), with respect to the ECG waveform 308 and the timeline 304 in order to designate which image frame from the sequence of image frames is currently being displayed on the display device 118. The icon 310 includes a dashed line 312 in order to help the user see how the position of the icon 310 corresponds to the ECG waveform 308. The user may use the user interface 115 to position the icon 310 at any point along the timeline 304 from T0 to T4 in order to select the image frame for display on the display device 118. The ECG waveform 308 provides an indication of the cardiac phase represented by the image frame currently being displayed on the display device 118. The user may, for instance, move the position of the icon 310 (such as with a trackball, touchpad, touchscreen, mouse, etc.) in order to scan through image frames in the sequence of image frames in order to identify an image frame representing a cardiac event. Additional information about the cardiac event will be provided hereinafter. After identifying the image frame representing the cardiac event, according to an exemplary embodiment, the clinician may use the user interface 115 in order to designate the target sequence. For example, according to the embodiment represented by FIG. 4, the clinician may use the user interface 115 to position a first marker 314 and a second marker 316 at appropriate places with respect to the ECG waveform 308 and the timeline 304 in order to designate the target sequence of image frames.

The cardiac event is a particular physiological event within the cardiac cycle for which it is desired to study more closely. Non-limiting examples of cardiac events include the following: mitral valve closure, mitral valve opening, aortic valve closure, aortic valve opening, tricuspid valve closure, tricuspid valve opening, pulmonary valve closure, pulmonary valve opening, initial ejection phase, peak ejection phase, early filling phase, atrial contraction, peak regurgitant flow area/volume, start of regurgitant flow, or end of regurgitant flow. Additional information regarding the embodiments related to regurgitant flow will be provided hereinafter. According to an exemplary embodiment, the clinician may select the target sequence so the target sequence includes a portion of the sequence of image frames before the cardiac event, a portion of the sequence of image frames after the cardiac event, and the image frame representing the cardiac event. The portion of the sequence of image frames before the cardiac event and the portion of the sequence after the cardiac event may both represent the same amount of time, or the portion of the sequence of image frames before the cardiac event and the portion of the sequence of image frames after the cardiac event may each represent a different amount of time. The clinician may identify the target sequence including a cardiac event that is different than the cardiac events listed hereinabove according to various other embodiments.

Regurgitant flow occurs when a valve is not fully closed (or not closed tightly enough) during a portion of the cardiac cycle when that particular valve should be tightly closed. As a result, the blood flows past this valve (which should have been closed tightly enough to eliminate any blood flow). The regurgitant flow is typically visualized in a color flow imaging mode as a jet or a stream flowing past the valve. Since regurgitant flow is indicative of some level of cardiac dysfunction, it is oftentimes of interest to the clinician. As such, it may be desirable to identify a target sequence of image frames showing the regurgitant flow. The processor 116 may be configured to identify the image frame with peak regurgitant flow (based on area or volume of the regurgitant flow) as representing the cardiac event according to various embodiments. According to other embodiments, the processor 116 may be configured to identify the first image frame with detectable regurgitant flow as representing the cardiac event, or the processor 116 may be configured to identify the last image frame with detectable regurgitant flow as representing the cardiac event according to various embodiments. According to an exemplary embodiment, the processor 116 may be configured to identify all of the image frame exhibiting regurgitant flow as the target sequence. Identifying the image frames showing regurgitant flow allows the processor 116 to reduce the playback speed of the image frames exhibiting regurgitant flow when displaying the sequence of image frames as a cine loop.

It may be desired to select a target sequence including the cardiac event of mitral valve opening according to an exemplary embodiment. On FIG. 4, time T2 represents the time of the mitral valve opening. As such, according to an exemplary embodiment, it may be desired to select a target sequence that includes the image frame associated with the time T2. As such, the user may use the user interface to position the first marker 314 at time T1 and the second marker 316 at the time T3. According to this exemplary embodiment, the target sequence includes a subset of the sequence of image frames from the time T1 to the time T3. In other words, the target sequence includes the subset of the sequence of image frames that were acquired between the time T1 to the time T3. As illustrated in FIG. 4, the target sequence from time T1 to the time T3 clearly includes the time T2, which is associated with the cardiac event of mitral valve opening according to this exemplary embodiment. According to various embodiments, the target sequence may represent a shorter length of time than either the systolic phase of the diastolic phase.

Additional details about embodiments that automatically, or semi-automatically identify the target sequence will be described hereinafter.

Referring back to FIG. 2, at step 206 the processor 116 displays the sequence of image frames as a cine-loop with multiple playback speeds on the display device 118. For purposes of this disclosure the terms “displaying the sequence of image frames as a cine-loop” and “displaying the cine-loop” will be afforded the same meaning. At step 206, the processor 116 is configured to playback the sequence of image frames so the target sequence of image frames is displayed at a slower playback speed than a portion of the sequence of images that is not part of the target sequence. The playback speed may be referred to in units such as “frames per second.” The target sequence is the portion of the sequence of image frames that was acquired between the times of T1 and T3. The portion of the sequence of image frames that is not part of the target sequence, according to the embodiment shown in FIG. 4, is the rest of the sequence of image frames—i.e., a first non-target sequence of image frames that was acquired from time T0 to time T1 and a second non-target sequence of image frames that was acquired from time T3 to T4. According to other embodiments where the target sequence of image frames extends either from the start of the sequence of image frames or extends to the end of the sequence of image frames, the portion of the sequence of image frames that is not part of the target sequence may be a single non-target sequence.

According to various embodiments, the slower playback speed may be 75% or less than the playback speed of the portion of the sequence of images that is not part of the target sequence. According to other embodiments, the slower playback speed may be 50% or less than the playback speed of the portion of the sequence of image frames that is not part of the target sequence. And according to other embodiments, the slower playback speed may be 25% or less than the playback speed of the portion of the sequence of image frames that is not part of the target sequence. The slower playback speed may be a different fraction of the playback speed of the portion of the sequence of images that is not part of the target sequence according to various embodiments.

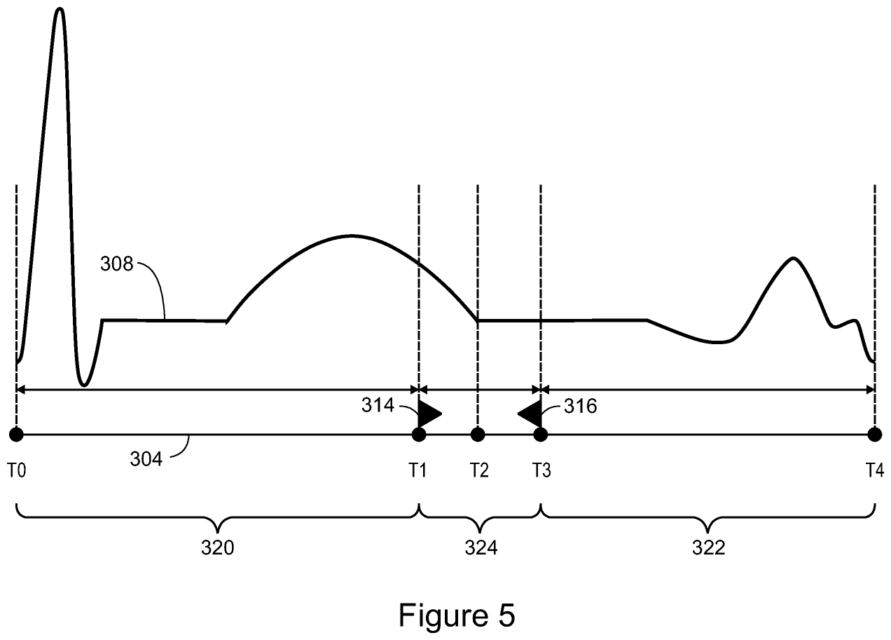

FIG. 5 is a representation of the ECG waveform 308 and the timeline 304. The ECG waveform 308 and the timeline 304 are the same as those shown in FIG. 4. As described with respect to FIG. 4, the target sequence of image frames is the subset of the sequence of image frames acquired between the times of T1 and T3 and includes the time T2, which is the time when the cardiac event of mitral valve opening occurs. At step 206, the processor 116 displays the sequence of images as a cine-loop. When displaying the sequence of image frames as a cine-loop, the processor 116 sequentially displays each image frame in the sequence of image frames. Since each image frame was acquired at a different time, and since the patient's heart was moving during the acquisition, displaying the sequence of image frames as a cine-loop illustrates motion of the heart that occurs between the time T0 and the time T4. The position of the ECG waveform 308 with respect to the ECG waveform 308 are exemplary and, as such, the position of the ECG waveform 308 may be different with respect to the times T0 and T4 according to various other embodiments. As discussed previously, this represents one complete cardiac cycle according to an embodiment, but it may represent more than one complete cardiac cycle according to other embodiments.

FIG. 5 also includes brackets to help illustrate the target sequence and the portion of the sequence of images that is not part of the target sequence. According to the exemplary embodiment shown in FIG. 5, the portion of the sequence of image frames that is not part of the target sequence includes two non-target sequences of image frames when represented as a single cardiac cycles as in FIG. 5.

FIG. 5 includes a first bracket 320 indicating a first non-target sequence of image frames, a second bracket 322 indicating a second non-target sequence of image frames, and a third bracket 324 indicating the target sequence of image frames. When displayed as a cine-loop, image frames representing the regions shown by the first bracket 320 and the second bracket 322 are displayed at a first playback speed and the image frames representing the region in the third bracket 324 are displayed at a second playback speed that is slower than the first playback speed.

Additionally, according to many embodiments, when displayed as a cine-loop, the sequence of image frames may be repeated multiple times. For example, when displaying the sequence of images as a cine-loop, after displaying the image frame acquired at the time T4, the processor 116 may next present the image frame of ultrasound data acquired at time T0. In this manner, according to an embodiment, the cine-loop may keep displaying the sequence of image frames until a command to stop is received via the user interface 115. According to the embodiment shown in FIG. 5, when displaying the cine-loop, the interval between displaying the last image frame acquired at the time T4 and the first image frame acquired at the time T0 may be the same as the interval between the other image frames in the first non-target sequence 320 and the second non-target sequence 322. Furthermore, it should be appreciated by those skilled in that art that, when displayed as a cine-loop, the second non-target sequence 322 will appear to be connected to the first non-target sequence 320.

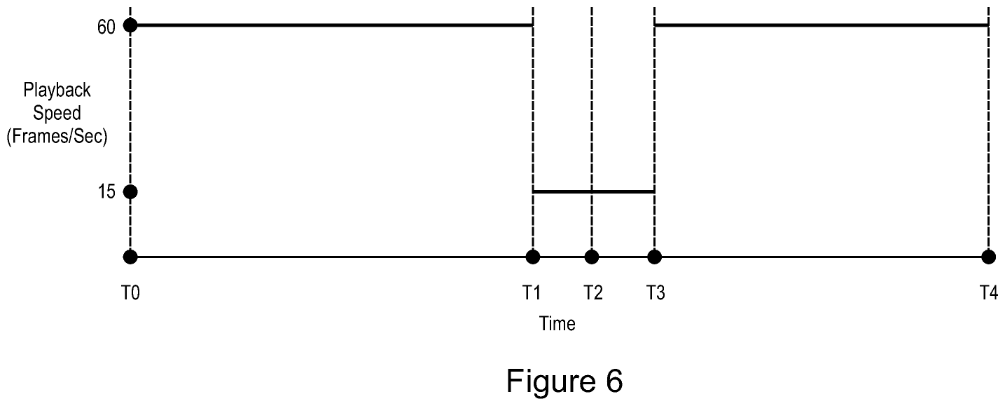

FIG. 6 is a graph of playback speed (in frames/second) versus time according to an exemplary embodiment. FIG. 6 illustrates the same embodiment described with respect to FIGS. 3, 4 and 5. In FIG. 6, from time T0 to T1, the playback speed may be 60 frames/second according to an embodiment. Likewise, from time T3 to T4, the playback speed is also 60 images/second. However, from time T1 to T3, the playback speed is only 15 frames/second, which is 25% of 60 frames/second. As discussed previously, the time T2 represents the cardiac event, which is a mitral valve opening according to an embodiment. By slowing down the playback speed of the cine-loop while displaying the image frames from time T1 to time T3, the clinician is able to more easily study the motion of the patient's heart associated with the cardiac event (i.e. mitral valve opening, according to an embodiment). Reducing the playback speed for image frames in the target sequence is effectively like displaying the image frames based on the target sequence in slow motion compared to the image frames representing one or more non-target sequences. As such, reducing the playback speed of the target sequence provides the clinician with the ability to study the motion associated with the cardiac event more carefully. Furthermore, reducing the playback speed of just the target sequence, as opposed to the entire sequence of image frames, clearly highlights the portion of the sequence of image frames that is most relevant with respect to a particular study or examination. As such, this makes it easier for the clinician when sharing the cine-loop with other colleagues since the key portion of the cine-loop, namely the target sequence of image frames, is being displaying with a slower playback speed than the rest of the image frames in the sequence of image frames.

FIG. 6 was used to describe an exemplary embodiment where there are two different playback speeds and there is no transition between the two different playback speeds. The graph of playback speed (in frames/second) versus time shown in FIG. 6 is therefore a step function. However, according to other embodiments, the processor 116 may be configured to provide a transition zone between playback speeds in order to make the appearance of the cine-loop on the display device more appealing to the clinician. For example, adding one or more transition zones may make displaying the sequence of image frames on the display device as a cine-loop appear smoother to the end user.

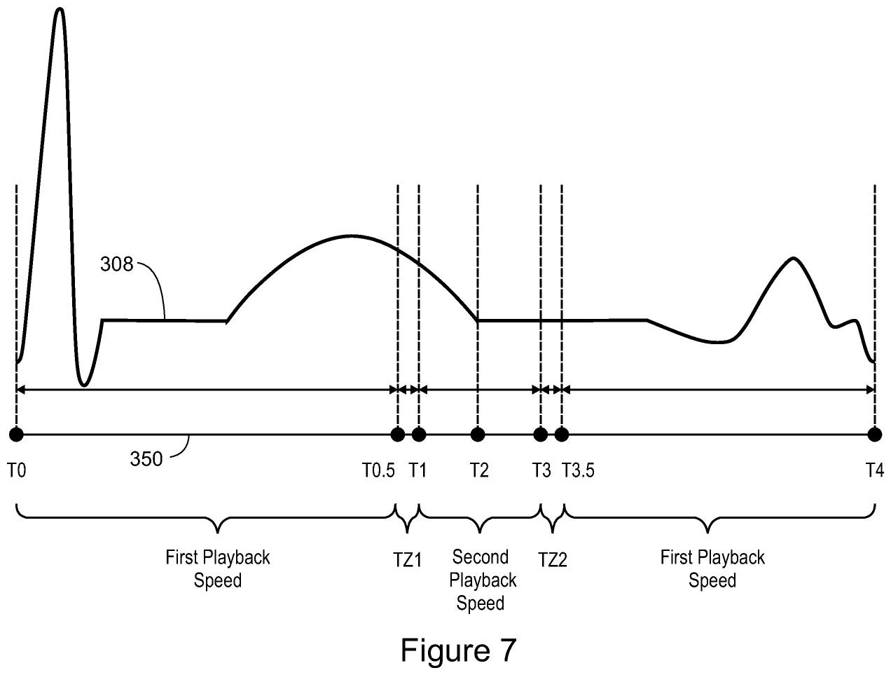

FIG. 7 is a representation of an ECG waveform and a timeline according to an exemplary embodiment. The ECG waveform 308 is the same as that shown in FIGS. 4 and 5. FIG. 7 includes a timeline 350 that is slightly different from the timelines shown with FIGS. 3, 4, 5, and 6. The timeline 350 includes the times T0, T1, T2, T3, and T4, which represent the same times with respect to the ECG waveform 308 as the times T0, T1, T2, T3, and T4 shown with respect to FIGS. 3, 4, 5, and 6. However, the timeline 350 also includes a time T0.5 and a time T3.5. According to the embodiment represented by FIG. 7, when displayed as a cine-loop, the portion of the sequence of image frames acquired from time from T0 to T0.5 and the portion of the sequence of image frames acquired from time T3.5 to T4 are both displayed at a first playback speed. The target sequence of image frames from the time T1 to the time T3 is displayed at a second playback speed that is slower than the first playback speed. The portion of the sequence of image frames from time T0.5 to time T1 is a first transition zone TZ1 and the portion of the sequence of image frames from time T3 to time T3.5 is a second transition zone TZ2. According to an exemplary embodiment, the processor 116 may adjust the playback speed during the first transition zone TZ1 and the second transition zone TZ2. The processor 116 may be configured to adjust the playback speed using a linear function, a polynomial function, a quadratic function, or any other type of function to transition between the first playback speed and the second playback speed and vice versa.

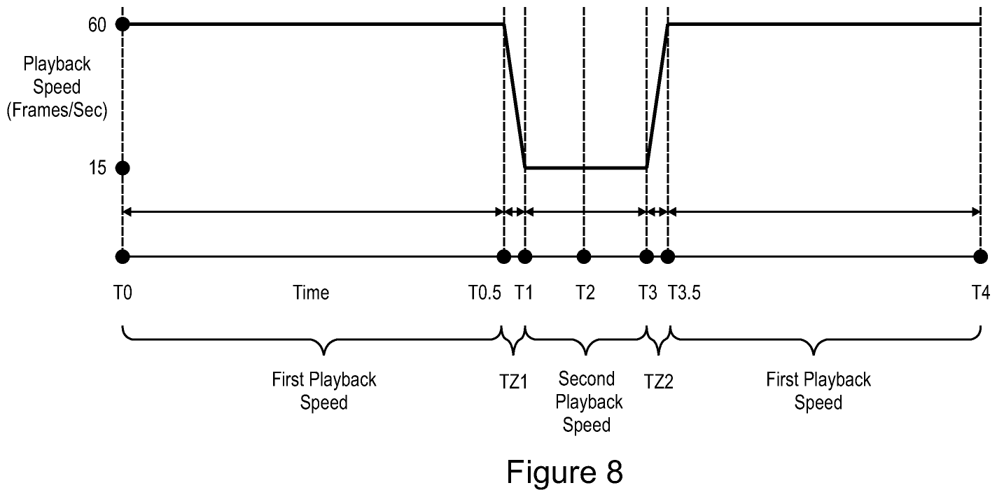

FIG. 8 is a graph of playback speed (in frames/second) versus time according to the embodiment that was discussed with respect to FIG. 7. Therefore, the timeline 350 shown in FIG. 8 is the same as the timeline 350 shown in FIG. 7. FIG. 8 illustrates an example where the processor 116 uses a linear function during the first transition zone TZ1 and the second transition zone TZ2. The graph shown in FIG. 8 illustrates an embodiment where the playback speeds are adjusted according to a linear function in both the first transition zone TZ1 and the second transition zone TZ2. For example, the portion of the sequence of image frames acquired from T0 to T0.5 is displayed at a first playback speed, which is 60 frames/second according to an exemplary embodiment. Likewise, the portion of the sequence of image frames acquired from T3.5 to T4 is also displayed at the first playback speed of 60 frames/second. The target sequence of image frames acquired from time T1 to time T3 are displayed at a second, slower, playback speed, which is 15 frames/second according to an embodiment. However, as evidenced by the graph in FIG. 8, the processor 116 adjusts the playback speed during the first transition zone TZ1 from 60 frames/second to 15 frames/second according to a linear function. And the processor 116 adjusts the playback speed during the second transition zone TZ2 from 15 frames/second to 60 frames/second according to another linear function. As discussed previously, providing one or more transitions zones, such as the first transition zone TZ1 and the second transition zone TZ2, may be used when displaying the sequence of images as a cine-loop in order to provide a smoother, less jarring, viewing experience for the clinician.

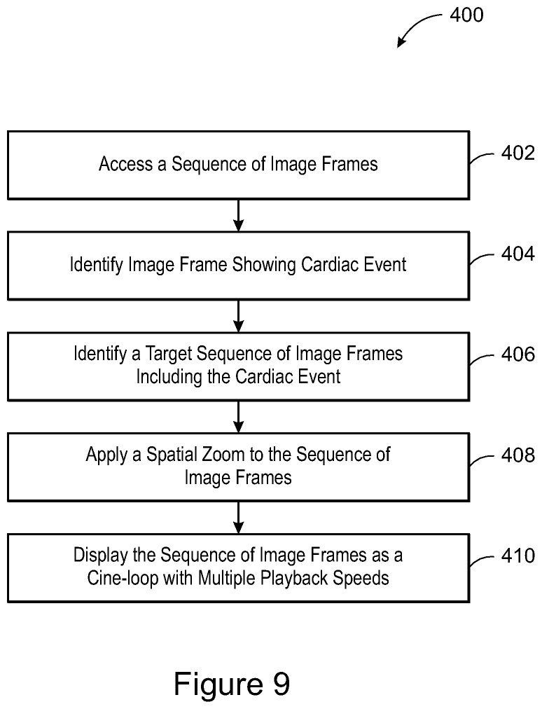

FIG. 9 is a flow chart of a method 400 in accordance with an exemplary embodiment. The individual blocks of the flow chart represent steps that may be performed in accordance with the method 400. Additional embodiments may perform the steps shown in a different sequence and/or additional embodiments may include additional steps not shown in FIG. 9. The technical effect of the method 400 is the displaying of a sequence of image frames as a cine-loop with multiple playback speeds, where a target sequence of image frames is displayed at a slower playback speed. The method 400 may be performed with the ultrasound imaging system 100 shown in FIG. 1. The method 400 will be described in detail hereinafter.

At step 402, the processor 116 accesses a sequence of image frames representing at least one cardiac cycle. Each of the image frames in the sequence of image frames may represent either a two-dimensional (2D) ultrasound image frame or a three-dimensional (3D) ultrasound image frame depending upon the type of ultrasound probe 106 and/or the imaging mode that was used during the acquisition. According to various other embodiments, some or all of the image frames may include color flow information (i.e., information acquired using a Doppler color flow mode). According to various embodiments, some or all of the image frames may not include color flow information. The processor 116 may access the sequence of image frames directly from the ultrasound probe 106. According to other embodiments, the processor 116 may access the sequence of image frames from a memory or a storage device. For example, the sequence of image frames may have been previously acquired and the processor 116 may access the sequence of image frames from memory or storage located on the ultrasound imaging system, such as the memory 120, or the processor 116 may access the sequence of image frames from a remote memory or a remote storage location. For example, the processor 116 may access the sequence of image frames from a different ultrasound imaging system, from a remote server, or from a Picture Archiving and Communications System (PACS), or from any other remote location.

Next, at step 404, the processor 116 identifies the image frame of the sequence of image frames showing the cardiac event. According to an exemplary embodiment, the processor 116 may implement at least one of an artificial intelligence technique in order to identify the image frame showing the cardiac event. According to an embodiment, the processor 116 may implement one or more trained neural networks (such as one or more convolutional neural networks) in order to identify the image frame showing the cardiac event. According to other embodiments, the artificial intelligence technique may include a machine learning technique that is used to identify the image frame showing the cardiac event. As discussed hereinabove, the image frame representing the cardiac event may be referred to as the target image frame. The processor 116 may be configured to identify the image frame by implementing any other type of artificial intelligence technique according to various embodiments, such as U-nets, Recurrent Neural Networks (RNNs), Transformers, or any other artificial intelligence techniques. According to other embodiments, the processor 116 may be configured to use image processing techniques, such as model-fitting, in order to identify the image frame showing the cardiac event. For example, the processor 116 may be configured to fit each image frame to a model of a heart and use the results to identify the cardiac event. According to other embodiments, the processor 116 may be configured to use a signal derived from color flow imaging/beams or tissue Doppler imaging/beams to identify the cardiac event. For example, the processor 116 may implement an artificial intelligence technique, such as one or more neural networks, that have been trained using color flow images and or tissue Doppler images to identify the cardiac event.

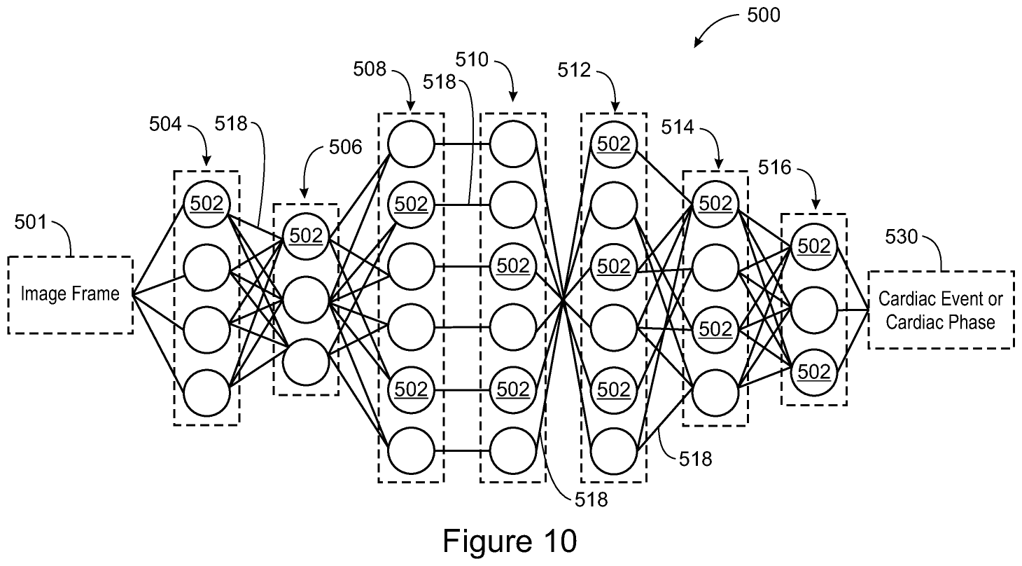

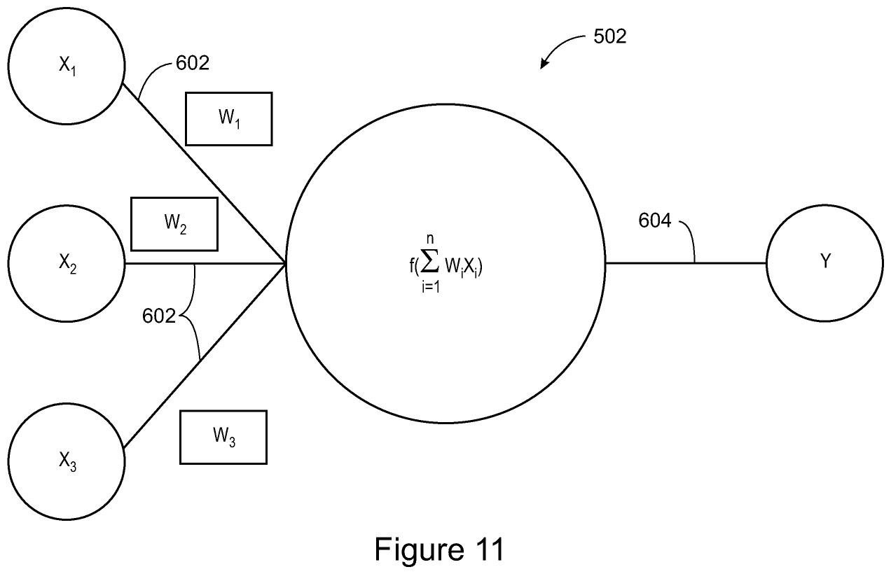

Referring now to FIGS. 10 and 11, an exemplary neural network for identifying a cardiac event from a sequence of image frames is shown. In some examples, the neural network may be trained with a training set of image frames.

FIG. 10 depicts a schematic diagram of a neural network 500 having one or more nodes/neurons 502 which, in some embodiments, may be disposed into one or more layers 504, 506, 508, 510, 512, 514, and 516. Neural network 500 may be a deep neural network. As used herein with respect to neurons, the term “layer” refers to a collection of simulated neurons that have inputs and/or outputs connected in similar fashion to other collections of simulated neurons. Accordingly, as shown in FIG. 10, neurons 502 may be connected to each other via one or more connections 518 such that data may propagate from an input layer 504, through one or more intermediate layers 506, 508, 510, 512, and 514, to an output layer 516.

FIG. 11 shows input and output connections for a neuron in accordance with an exemplary embodiment. As shown in FIG. 10, connections (e.g., 518) of an individual neuron 502 may include one or more input connections 602 and one or more output connections 604. Each input connection 602 of neuron 502 may be an output connection of a preceding neuron, and each output connection 604 of neuron 502 may be an input connection of one or more subsequent neurons. While FIG. 11 depicts neuron 502 as having a single output connection 604, it should be understood that neurons may have multiple output connections that send/transmit/pass the same value. In some embodiments, neurons 502 may be data constructs (e.g., structures, instantiated class objects, matrices, etc.), and input connections may be received by neuron 502 as weighted numerical values (e.g., floating point or integer values). For example, as further shown in FIG. 11, input connections X1, X2, and X3 may be weighted by weights W1, W2, and W3, respectively, summed, and sent/transmitted/passed as output connection Y. As will be appreciated, the processing of an individual neuron 502 may be represented generally by the equation:

$$Y = f ( {\sum }_{i = 1}^{n} {W}_{i} {X}_{i} )$$

where n is the total number of input connections 602 to neuron 502. In one embodiment, the value of Y may be based at least in part on whether the summation of WiXi exceeds a threshold. For example, Y may have a value of zero (0) if the summation of the weighted inputs fails to exceed a desired threshold.

As will be further understood from FIGS. 10 and 11, input connections 602 of neurons 502 in input layer 504 may be mapped to an input 501, while output connections 604 of neurons 502 in output layer 516 may be mapped to an output 530. As used herein, “mapping” a given input connection 602 to input 501 refers to the manner by which input 501 affects/dictates the value said input connection 602. Similarly, as also used herein, “mapping” a given output connection 604 to output 530 refers to the manner by which the value of said output connection 604 affects/dictates output 530.

Accordingly, in some embodiments, the acquired/obtained input 501 is passed/fed to input layer 504 of neural network 500 and propagated through layers 504, 506, 508, 510, 512, 514, and 516 such that mapped output connections 604 of output layer 516 generate/correspond to output 530. As shown, input 501 may include an image frame. The image frame may depict a one or more structures that are identifiable by the neural network 500. The neural network may also detect if the image frame is/includes the cardiac event. Further, output 530 may include locations and contours for the one or more structures that are identified by the neural network 500.

Neural network 500 may be trained using a plurality of training datasets. Each training dataset may include image frames that are, for example, annotated. Based on the training datasets, the neural network 500 may learn to identify a cardiac phase or a cardiac event represented by each of the image frames. According to an embodiment, the plurality of training datasets may be arranged by view and annotated to designate the cardiac phase represented in each image frame used for training and/or the cardiac event represented in each image frame used for training. A non-limiting list of examples of cardiac events includes the following: mitral valve closure, mitral valve opening, aortic valve closure, aortic valve opening, tricuspid valve closure, tricuspid valve opening, pulmonary valve closure, pulmonary valve opening, initial ejection phase, peak ejection phase, early filling phase, and atrial contraction. A non-limiting list of views that may be used during the training includes both apical views and parasternal views. A non-limiting list of apical views includes a four-chamber view (4CH), a two-chamber view (2CH), an apical long-axis view (APLAX), a five-chamber view (5CH), and apical view with right ventricle focus (A-RV). A non-limiting list of parasternal views includes: a parasternal long-axis view (PLAX), and a parasternal short-axis view (PSAX). Additionally, there are at least three different variants of PLAX views and at least 4 different variants of PSAX views. Depending upon the differences between each of the standard views, it may be necessary to train the neural network using training image frames from each of the standard views and for each of the cardiac phases and/or cardiac events for which the neural network is intended to be used. The machine learning, or deep learning, therein (due to, for example, identifiable trends in placement, size, etc. of features associated with a cardiac phase or cardiac event) may cause weights (e.g., W1, W2, and/or W3) to change, input/output connections to change, or other adjustments to neural network 500. Further, as additional training datasets are employed, the machine learning may continue to adjust various parameters of the neural network 500 in response. As such, a sensitivity of the neural network 500 may be periodically increased, resulting in a greater accuracy of cardiac phase identification and/or cardiac event identification. FIG. 10 is a schematic representation of an exemplary neural network. It should be appreciated by those skilled in the art that neural networks of other configurations may be used according to various embodiments.

Referring back to the method 400 shown in FIG. 9, at step 404, the processor 116 identifies one of the sequence of image frames showing the cardiac event. According to various embodiments, the user may be able to select the cardiac event that is desired from a plurality of cardiac events. The processor 116 may be configured to identify which of the sequence of image frames includes the cardiac event by implementing an artificial intelligence technique, such as one more neural networks. According to another embodiment, the processor 116 may be configured to implement the artificial intelligence technique, such as one or more neural networks, in order to identify a cardiac phase associated with each image frame in the sequence of image frames. After identifying a cardiac phase associated with each image frame in the sequence of image frames, the processor 116 may identify the cardiac phase associated with the desired cardiac event. According to other embodiments, the processor 116 may be configured to implement the artificial intelligence technique in order to identify the cardiac event without identifying the cardiac phase. For example, the processor 116 may identify which of the sequence of image frames represents the cardiac event just by using the artificial intelligence technique, such as by implementing one or more trained neural networks.

According to an embodiment, after identifying the image frame including the cardiac event (i.e., the target image frame), at step 406 the processor 116 identifies the target sequence of image frames from the sequence of image frames. As discussed previously, the target sequence of image frames is a subset of the sequence of image frames including the image frame showing the cardiac event identified at step 404. For most embodiments, the target sequence will include a first plurality of images frames acquired within a first predetermined amount of time before the cardiac event, a second plurality of image frames acquired within a second predetermined amount of time after the cardiac event, and the target image frame representing the cardiac event. According to various embodiments, the first predetermined amount of time and the second predetermined amount of time may both be preset values. Or the first predetermined amount of time and the second predetermined amount of time may both be user configurable. The clinician, may, for instance, use the user interface 115 in order to select the length of time for the first predetermined amount of time and the second predetermined amount of time. According to these embodiments, the clinician may therefore control the amount of time represented by the target sequence of image frames.

According to other embodiments, the target sequence of image frames may only include one of the first plurality of image frames acquired within the first predetermined amount of time before the cardiac event or the second plurality of image frames acquired within the second predetermined amount of time after the cardiac event. In other words, the cardiac event may be at either the start of the target sequence or the end of the target sequence according to various embodiments. However, for many embodiments, it may be desirable to have context from image frames acquired both from before the cardiac event and from after the cardiac event.

At step 408, the processor 116 applies a spatial zoom to the sequence of image frames in order to zoom-in on an anatomical structure. For example, the processor 116 may use image processing techniques or artificial intelligence techniques in order to identify one or more anatomical structures within the image frame showing the cardiac event and/or other image frames in the target sequence of image frames. For example, the processor 116 may use any type of image processing technique to identify the one or more anatomical structures. For instance, the processor 116 may use edge detection, B-splines, shape-based detection algorithms, average intensity, segmentation, speckle tracking, or any other image-processing based techniques to identify one or more anatomical structures. According to other embodiments, the processor 116 may implement an artificial intelligence technique in order to identify the anatomical structure within the image frame including the cardiac event and/or one or more other image frames in the target sequence. For example, the processor 116 may implement one or more neural networks that have been trained to identify the anatomical structure.

After identifying the anatomical structure, the processor 116 may be configured to automatically apply a spatial zoom to the target sequence of image frames. For example, the spatial zoom may be applied so the anatomical structure fills a specific percentage of the view. Or the processor 116 may apply the spatial zoom so the anatomical feature is a target size when the target sequence is displayed. Step 408 is optional. Some embodiments may not involve applying spatial zoom after identifying the target sequence. However, for embodiments that do implement step 408, automatically applying spatial zoom to the target sequence of images may provide an easier viewing experience for the clinician. Automatically applying a spatial zoom helps focus the clinician's attention on the anatomical structure and shows the motion associated with the cardiac event in the target sequence of image frames in greater detail.

At step 410, the processor is configured to display the sequence of image frames as a cine-loop with multiple playback speeds. As discussed with respect to the method 200, the processor 116 may be configured to playback the sequence of image frames so the target sequence of image frames is displayed at a slower playback speed than a portion of the sequence of images that is not part of the target sequence. Since the step of displaying the sequence of image frames as a cine-loop with multiple playback speeds was described with respect to step 206 of the method 200, it will not be described in detail with respect to the method 400.

According to an embodiment, a method of echocardiography includes accessing a sequence of image frames representing at least one cardiac cycle, wherein each of the image frames in the sequence of image frames is based on ultrasound data. The method includes identifying a target sequence of image frames including a cardiac event, wherein the target sequence of image frames is a subset of the sequence of image frames. And the method includes displaying the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed

According to an embodiment, the portion of the sequence of image frames that is not part of the target sequence comprises a non-target sequence.

According to an embodiment, the portion of the sequence of image frames that is not part of the target sequence comprises a first non-target sequence and a second non-target sequence, wherein the first non-target sequence is before the target sequence in the sequence of image frames and the second non-target sequence is after the target sequence in the sequence of image frames.

According to an embodiment, the second playback speed is 50% or less of the first playback speed.

According to an embodiment, the second playback speed is 25% or less of the first playback speed.

According to an embodiment, identifying the target sequence of image frames is performed automatically by the at least one processor.

According to an embodiment, implements an artificial intelligence technique in order to identify the target sequence of image frames.

According to an embodiment, at least one processor implements an artificial intelligence technique in order to identify a cardiac phase associated with each image frame in the sequence of image frames in order to identify the cardiac event, and wherein the at least one processor identifies the target sequence as including a first plurality of image frames acquired within a first predetermined amount of time before the cardiac event, a second plurality of image frames acquired within a second predetermined amount of time after the cardiac event, and a target image frame including representing the target event.

According to an embodiment, the method comprises automatically applying a spatial zoom to the sequence of image frames in order to zoom-in on an anatomical structure while displaying the sequence of image frames on the display device as the cine-loop.

According to an embodiment, the cardiac event is selected from the following list: an opening of a valve, a closing of a valve, an initial ejection phase, a peak ejection phase, an early filling phase, an atrial contraction phase, and a septal flash.

According to an embodiment, an ultrasound imaging system for echocardiography includes an ultrasound probe, a display device and at least one processor in electronic communication with the ultrasound probe and the display device. The at least one process is configured to access a sequence of image frames representing at least one cardiac cycle, wherein each of the image frames in the sequence of image frames is based on ultrasound data. The at least one processor is configured to identify a target sequence of image frames including a cardiac event, wherein the target sequence of image frames is a subset of the sequence of image frames. The at least one processor is configured to display the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of echocardiography comprising:
accessing a sequence of image frames representing at least one cardiac cycle, wherein each of the image frames in the sequence of image frames is based on ultrasound data;
identifying a target sequence of image frames including a cardiac event, wherein the target sequence of image frames is a subset of the sequence of image frames; and
displaying the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed.

2. The method of claim 1, wherein the portion of the sequence of image frames that is not part of the target sequence comprises a non-target sequence.

3. The method of claim 1, wherein the portion of the sequence of image frames that is not part of the target sequence comprises a first non-target sequence and a second non-target sequence, wherein the first non-target sequence is before the target sequence in the sequence of image frames and the second non-target sequence is after the target sequence in the sequence of image frames.

4. The method of claim 1, wherein the second playback speed is 50% or less of the first playback speed.

5. The method of claim 1, wherein the second playback speed is 25% or less of the first playback speed.

6. The method of claim 1, wherein said identifying the target sequence of image frames is performed automatically by at least one processor.

7. The method of claim 6, wherein the at least one processor implements an artificial intelligence technique in order to identify the target sequence of image frames.

8. The method of claim 7, wherein the at least one processor implements the artificial intelligence technique in order to identify a cardiac phase associated with each image frame in the sequence of image frames in order to identify the cardiac event, and wherein the at least one processor identifies the target sequence as including a first plurality of image frames acquired within a first predetermined amount of time before the cardiac event, a second plurality of image frames acquired within a second predetermined amount of time after the cardiac event, and a target image frame representing the cardiac event.

9. The method of claim 1, further comprising automatically applying a spatial zoom to the sequence of image frames in order to zoom-in on an anatomical structure while displaying the sequence of image frames on the display device as the cine-loop.

10. The method of claim 1, wherein the cardiac event is selected from the following list: an opening of a valve, a closing of a valve, an initial ejection phase, a peak ejection phase, an early filling phase, an atrial contraction phase, and a septal flash.

11. An ultrasound imaging system comprising:
an ultrasound probe;
a display device; and
at least one processor in electronic communication with the ultrasound probe and the display device, wherein the at least one processor is configured to:
access a sequence of image frames representing at least one cardiac cycle, wherein each of the image frames in the sequence of image frames is based on ultrasound data;
identify a target sequence of image frames including a cardiac event, wherein the target sequence of image frames is a subset of the sequence of image frames; and
display the sequence of image frames on the display device as a cine-loop with multiple playback speeds, where a portion of the sequence of image frames that is not part of the target sequence of image frames is displayed at a first playback speed, and where the target sequence of image frames is displayed at a second playback speed that is slower than the first playback speed.

12. The system of claim 11, wherein the portion of the sequence of image frames that is not part of the target sequence comprises a non-target sequence.

13. The system of claim 11, wherein the portion of the sequence of image frames that is not part of the target sequence comprises a first non-target sequence and a second non-target sequence, wherein the first non-target sequence is before the target sequence in the sequence of image frames and the second non-target sequence is after the target sequence in the sequence of image frames.

14. The system of claim 11, wherein the second playback speed is 50% or less of the first playback speed.

15. The system of claim 11, wherein the second playback speed is 25% or less of the first playback speed.

16. The system of claim 11, wherein the at least one processor is configured to automatically identify the target sequence of image frames.

17. The system of claim 16, wherein the at least one process is configured to implement an artificial intelligence technique in order to identify the target sequence of image frames.

18. The system of claim 17, wherein the at least one processor is configured to implement the artificial intelligence technique in order to identify a cardiac phase associated with each image frame in the sequence of image frames in order to identify the cardiac event, and wherein the at least one processor identifies the target sequence as including a first plurality of image frames acquired within a first predetermined amount of time before the cardiac event, a second plurality of image frames acquired within a second predetermined amount of time after the cardiac event, and a target image frame representing the cardiac event.

19. The system of claim 11, wherein the processor is further configured to apply a spatial zoom to the sequence of image frames in order to zoom-in on an anatomical structure while displaying the sequence of image frames on the display device as the cine loop.

20. The system of claim 11, wherein the cardiac event is selected from the following list: an opening of a valve, a closing of a valve, an initial ejection phase, a peak ejection phase, an early filling phase, an atrial contraction phase, and a septal flash.
